(19) United States
(12) Patent Application Publication
(10) Pub. No.: US 2023/0131960 A1
(43) Pub. Date: Apr. 27, 2023

(54) NEW STRATEGY TO TREAT AND PREVENT DISEASES CAUSED BY ENTEROBACTERIAE

(71) Applicants: INSERM (Institut National de la Santé et de la Recherche Médicale), Paris (FR); Université de Rennes 1, Rennes (FR)

(72) Inventors: Latifa BOUSARGHIN, Rennes Cedex 9 (FR); Olivier LOREAL, Rennes Cedex 9 (FR); Zohreh TAMANAI-SHACOORI, Rennes Cedex 9 (FR); Anne JOLIVET-GOUGEON, Rennes Cedex 9 (FR); Sandrine DAVID, Rennes Cedex 9 (FR)

(21) Appl. No.: 17/906,268

(22) PCT Filed: Mar. 24, 2021
(86) PCT No.: PCT/EP2021/057566

§ 371 Date: Sep. 14, 2022

(30) Foreign Application Priority Data
Mar. 25, 2020 (EP) 20305316.0

(51) Int. Cl. A61K 35/741; A61P 31/04

(52) U.S. Cl. CPC A61K 35/741; A61P 31/04; A61K 2035/115

(57) ABSTRACT

The present invention relates to the treatment of diseases induced by Enterobacteriae. The inventors evaluated, in a multicellular in vitro model associating cells representing human enterocytes (Caco-2 cells), goblet mucus secreting cells (HT29-MTX) and M cells, whether Bacteroides fragilis, a non-enterotoxigenic strain, could be useful to limit the severity of the Salmonella Heidelberg infection, with an hypermutator phenotype, by analyzing their impact on growth and mucosal translocation. Thus, the present invention relates to a Bacteroides fragilis strain for use in the treatment of diseases induced by Enterobacteriae in a subject in need thereof.

### FIELD OF THE INVENTION

The present invention relates to a Bacteroides fragilis strain for use in the treatment of diseases induced by Enterobacteriae in a subject in need thereof.

### BACKGROUND OF THE INVENTION

The intestinal epithelium is composed of multiple cell types including enterocytes, the cells involved in digestive absorption, goblet cells that produce the mucus recovering the luminal face of enterocytes and Microfold cells (M cells) specialized in antigen sampling. The surface of this epithelium can be exposed to a large variety of harmful pathogens which can gain access to the lamina propria. Thus, bacteria have been observed within the basal paracellular space of polarized enterocyte monolayers [1], [2]. Translocation of bacteria across the intestinal epithelium may occur via a transcellular route, involving an endocytic uptake followed by intracellular trafficking. In addition, translocation of intestinal microflora can occur through M cells which possess a high phagocytic and transcytotic capacity [3]. Several pathogens such as Salmonella, Shigella and Yersinia exploit M cells to invade mucosal tissues and cross the digestive epithelial barrier before reaching the bloodstream [4], [5], [6].

Among Salmonella, the Salmonella Heidelberg (S. Heidelberg) is one of the most common serovar causing severe extra-intestinal infections [7]. Within the natural population of S. Heidelberg, some strains display a hypermutator phenotype related to the frequent occurrence of mutations in the genes involved in methyl mismatch repair system [8], [9]. The hypermutator phenotype allows bacteria to adapt to adverse and stringent environmental conditions including the pressure of antibiotic exposure [10]. Some hypermutator bacteria are multidrug resistant, there is an urgent need to develop new therapeutic alternatives.

The use of probiotics has been suggested as a potential new strategy to limit the development and/or severity of digestive bacterial infection by decreasing pathogen load [11], [12]. Approved probiotics are based on intestinal microbiota species including Lactobacillus and Bifidobacterium [13]. Interestingly, no representatives of the Bacteroidetes phylum, one of the major component of the intestinal flora, have been proposed. Among the Bacteroides species, Bacteroides fragilis represents a major anaerobe that is a commensal colonizer of the mammalian lower gastrointestinal tract [14], despite the fact that some enterotoxigenic B. fragilis strains exist due to a B. fragilis pathogenicity island (BfPAI). Importantly, some studies raising the possibility that the non-enterotoxigenic B. fragilis strains found in intestines of healthy individuals could be used as probiotic therapy [15].

### SUMMARY OF THE INVENTION

The inventors evaluated, in a multicellular in vitro model associating cells representing human enterocytes (Caco-2 cells), goblet mucus secreting cells (HT29-MTX) and M cells, whether Bacteroides fragilis, a non-enterotoxigenic strain, could be useful to limit the severity of the Salmonella Heidelberg infection, with an hypermutator phenotype, by analyzing their impact on growth and mucosal translocation. Thus, the invention relates to a Bacteroides fragilis strain for use in the treatment of diseases induced by Enterobacteriae in a subject in need thereof. Particularly, the invention is defined by its claims

### DETAILED DESCRIPTION OF THE INVENTION

### Bacteroides fragilis and Use Thereof

A first object of the invention relates to a Bacteroides fragilis strain for use in the treatment of diseases induced by an Enterobacteriae in a subject in need thereof.

As use herein, the term Enterobacteriae denotes a large family of Gram-negative bacteria. Enterobacteriaceae includes, along with many harmless symbionts, many of the more familiar pathogens, such as Salmonella like Salmonella Heidelberg, Escherichia coli, Yersinia pestis, Klebsiella, and Shigella. Other disease-causing bacteria in this family include Proteus, Enterobacter and Citrobacter.

In a particular embodiment, the invention relates to a Bacteroides fragilis strain for use in the treatment of diseases induced by Salmonella or Escherichia coli in a subject in need thereof.

In a particular embodiment, the invention also relates to at least one Bacteroides fragilis strain for use in the treatment of diseases induced by an Enterobacteriae in a subject in need thereof.

In a particular embodiment, the invention also relates to a Bacteroides fragilis strain for use in a veterinary treatment of diseases induced by Enterobacteriae in a subject in need thereof.

In a more particular embodiment, the invention relates to a Bacteroides fragilis strain for use in a veterinary treatment in fish.

Another aspect of the invention relates to a Bacteroides fragilis strain for use in the treatment of an Inflammatory Bowel Disease (IBD) and/or an Irritable Bowel Syndrome (IBS) in a subject in need thereof.

In some embodiments, the Inflammatory Bowel Disease is a Crohn's disease or an ulcerative colitis.

As used herein, the term “Bacteroides fragilis” denotes an obligately anaerobic, Gram-negative, rod-shaped bacterium. The ATCC number for the B. fragilis that was used is: ATCC 25285 but NTBF TM 4000 (clinical isolate Pasteur Institut, M. Sebal), YCH46 [43] strains could be also used. Other Bacteroides such as Bacteroides Vulgaris [44] and Bacteroides thetaiotaomicron [38] could be considered. It is part of the normal microbiota of the human colon and is generally commensal but can cause infection if displaced into the bloodstream or surrounding tissue following surgery, disease, or trauma.

In some embodiments, the Bacteroides fragilis strain is a non-toxigenic strain. As example, the non-toxigenic strain may be NTBF TM 4000, YCH46, LM3, LM9 et LM59 (Mundy and Sears 1996).

In a particular embodiment, the Bacteroides fragilis strain is used as a probiotic.

As used herein, the Bacteroides fragilis strain can be understand in singular and plural (Bacteroides fragilis bacteria).

According to the invention, the Bacteroides fragilis strain can be ingested live or not in adequate quantities to exert beneficial effects on the human health and particularly to treat diseases or infections induced by Enterobacteriae like Salmonella Heidelberg and E. coli in a subject in need thereof.

In a particular embodiment, the invention relates to a Bacteroides fragilis strain for use for inhibiting the Enterobacteriae translocation. In a more particular embodiment, the Enterobacteriae translocation is a Salmonella and E. coli translocation.

In particular embodiment, Bacteroides fragilis strain is cultured in an appropriate medium and the supernatant obtained after culture is administrated to a subject in need thereof.

Thus, the invention also relates to a supernatant obtained after culture of Bacteroides fragilis strain for use in the treatment of diseases induced by Enterobacteriae in a subject in need thereof.

Particularly, the invention relates to a supernatant obtained after culture of Bacteroides fragilis strain for use in the treatment of diseases induced by Salmonella or Escherichia coli in a subject in need thereof.

In a particular embodiment, the supernatant of Bacteroides fragilis strain may be obtainable by a method comprising the following steps of a) providing Bacteroides fragilis strain, b) culturing the bacteria in an appropriate medium, particularly DMEM medium c) optionally washing the cells from step a) and b), e) separating the supernatant from the bacteria.

Particularly, the step of separation can be done by using a 0, 22 μm filter.

In a particular embodiment, the supernatant may be “inactivated” prior to use, for example by irradiation. Therefore, the method for preparing the supernatant may comprise an optional additional irradiation step f).

As used herein the term “probiotic” denotes live microorganisms intended to provide health benefits when consumed, generally by improving or restoring the gut flora.

As used herein, the term “diseases induced by Enterobacteriae” denotes a group of disease including but not limited to Salmonellosis, typhoid fever, diarrhea Crohn's disease, travelers' diarrhea or ulcerative colitis. The term induced by Enterobacteriae as to be interpreted as due to a colonization of Enterobacteriae in the gastrointestinal tract, which is responsible of an aggravation of the disease. To determine if a disease is induced or not by Enterobacteriae, many factors (host genetics, the complex gut tissue environment, microbial dysbiosis, impaired gut barrier function, and dysregulated innate/adaptive immune system) drive the pathogenic immune response and underlie the emphatic failure to resolve gut inflammation in IBD.

As used herein, the term “diseases induced by Salmonella Heidelberg” denotes a group of disease induced by Enterobacteriae like Salmonellosis, travelers ‘diarrhea or typhoid fever.

As used herein, the term diseases “induced by Escherichia coli” denotes a group of disease induced by Enterobacteriae like diarrhea, Crohn's disease and ulcerative colitis.

As used herein, the term “Inflammatory Bowel Disease (IBD)” denotes a disorder involving chronic inflammation of digestive tract and/or destruction of the bowel wall. Inflammatory bowel disease (IBD) in humans, such as Crohn's disease and ulcerative colitis, is a complex chronic inflammatory disorder of largely unknown cause in a genetically predisposed host. The contributions of the host immune system and the genetic factors that predispose to IBD have been extensively researched and recently reviewed. It has also been hypothesized that a breakdown in the balance between putative protective species and “harmful” species could contribute to IBD pathogenesis [40]. For instance, many studies have documented reduced bacterial diversity and richness in IBD patients, largely due to decrease of firmicutes and increase of Bacteroidetes phyla [42] [43] [47] [45].

Lo Presti et al. 2019 [41] showed that Enterobacteriaceae and Streptococcus were associated to IBD microbiota. Regarding Enterobacteriaceae, previous studies have found elevated abundance of this family in Crohn's Disease patients [39] [46], supporting these data and that the Gammaproteobacteria [38] (e.g., E. coli AIEC strain, Klebsiella spp., Pseudomonas spp., and Salmonella) overgrew in mucosa of IBD patients [44]. IBD may be induced or not by Enterobacteriae.

As used herein, the term “Irritable Bowel Syndrome (IBS)” is a functional gastrointestinal disorder. IBS usually causes no ulcers or lesions in the bowel and may be induced or not by Enterobacteriae.

As used herein, the term “subject” denotes a mammal, such as a rodent, a feline, a canine, an ovine, a bovine, a pork and a primate. Particularly, the subject according to the invention is a human. More particularly, the subject according to the invention is a subject infected with Salmonella Heidelberg with no symptoms of a disease caused by Salmonella Heidelberg.

As used herein, the term “veterinary treatment” denotes the prevention, the alleviation or the eradication of a symptom in an animal. In a particular embodiment, the animal is a mammal, such as a rodent, a feline, a canine, an ovine, a bovine, a pork and a primate. Fish farming can be also considered. Numerous studies show that fish can have a disruption of their intestinal microbiota because of for example water contaminated by pollutants. Indeed, it was shown that ammonia exposure could induce the immune response in crucian carp, and alter the gut microbial community. Some studies have reported that aquatic animals can increase the rate of infection of exogenous pathogens and mortality after exposure to ammonia. It has been shown that in fish there are Salmonella such as Salmonella Weltevreden which is the most common serovar isolated in both aquaculture systems and the closely related genotypes suggests that this serovar may have increased ability to survive and even multiply in tropical aquatic environments [41]. Ammonia could also affect the abundance of bacteroides in fish gut, where previous reports have shown that there are three phyla of dominant bacteria in the intestinal tract of common carp: Fusobacteria, Proteobacteria and Bacteroidetes [42]. So Bacteroides fragilis is useful in fish, it can use a variety of food carbohydrates as energy. Besides other probiotics such as Bacillus cereus and lactobacillus were used as probiotics at the protein level in fish [40] [39]. It was shown that Bacillus cereus strain QSI-1 can decrease the pathogenicity of Aeromonas hydrophila YJ-1 in zebrafish and Goldfish models.

Thus, the invention also relates to a Bacteroides fragilis strain for use in the treatment of diseases induced by Enterobacteriae such as Salmonella or Escherichia coli in a subject with no symptoms of a disease caused by Salmonella Heidelberg.

A method for treating diseases induced by an Enterobacteriae comprising administering to a subject in need thereof a therapeutically effective amount of a Bacteroides fragilis strain.

### Composition and Use Thereof

A second object of the invention relates to a therapeutic composition comprising Bacteroides fragilis strain according to the invention.

In another embodiment, the invention also relates to a therapeutic composition comprising the supernatant according to the invention.

In a particularly embodiment, the invention relates to a therapeutic composition comprising a Bacteroides fragilis strain for use in the treatment of diseases induced by Enterobacteriae such as Salmonella or Escherichia coli in a subject in need thereof.

In a particular embodiment, the therapeutic composition according to the invention is intended for mucosal administration to a subject.

In another particular administration, the therapeutic composition according to the invention is intended for oral administration to a subject in need thereof. For example, compositions can be in the form of a suspension, tablet, pill, capsule, granulate or powder.

In a liquid therapeutic composition, the Bacteroides fragilis strain of the invention is present, free and not immobilized, in suspension. The suspension has a composition which ensures physiological conditions for the bacteria, so that in particular the osmotic pressure within the cell does not lead to lysis.

In a solid therapeutic composition, the Bacteroides fragilis strain according to the invention can be present in free, particularly lyophilized form, or in immobilized form. For example, the Bacteroides fragilis strain according to the invention can be enclosed in a gel matrix which provides protection for the cells.

A solid therapeutic composition intended for oral administration and containing the Bacteroides fragilis strain according to the invention in immobilized or non-immobilized form is particularly provided with a coating resistant to gastric juice. It is thereby ensured that the food-grade bacterium contained in the therapeutic composition can pass through the stomach unhindered and undamaged and the release of the Bacteroides fragilis strain first takes place in the upper intestinal regions.

In another aspect of the invention, the therapeutic composition contains sufficient colony-forming units (CFU) of the Bacteroides fragilis strain so that with multiple administration of the therapeutic composition to a subject in need thereof, the state of the diseases induced by Salmonella Heidelberg, the progression of diseases induced by Salmonella Heidelberg are stopped, and/or the symptoms of the diseases induced by Salmonella Heidelberg can be alleviated. According to the invention, it is in particular provided that a therapeutic composition contains for example 1×10⁸-1×10¹¹, particularly 1×10⁹-1×10¹⁰ CFU of the Bacteroides fragilis strain according to the invention.

In a further preferred embodiment of the invention, the therapeutic composition containing the Bacteroides fragilis strain is administered intrarectally. A rectal administration particularly takes place in the form of a suppository, enema or foam.

In another aspect, the invention relates to a food composition comprising the Bacteroides fragilis strain according to the invention.

In a particular embodiment, food compositions according to the invention are intended for oral administration to a subject. For example, compositions can be in the form of a suspension, tablet, pill, capsule, granulate, powder or yogurt.

In a preferred embodiment, the food composition may contain for example 1×10⁸-1×10¹¹, particularly 1×10⁹-1×10¹⁰ CFU of the Bacteroides fragilis strain according to the invention.

In a preferred embodiment, the food composition may be administered to the subject in need thereof for example at a daily dose of 10¹⁰ bacteria.

The invention will be further illustrated by the following figures and examples. However, these examples and figures should not be interpreted in any way as limiting the scope of the present invention.

### FIGURES

FIG. 1: Epithelial integrity during cells differenciation of Caco-2, Caco-2/HT29 MTX (double co-culture), Caco-2/HT29 MTX/M (triple co-culture): (A) Transepithelial electrical resistance (TEER) analysis; (B) Lucifer Yellow (LY) permeability crossing different monolayers, expressed in pmol/cm2/s in basal compartment (*p<0.05).

FIG. 2: Interaction between the in vitro co-culture models (Caco-2, double and triple) with bacteria (Salmonella Heidelberg (Salmonella) and Bacteroides fragilis (B.fragilis)): translocation of bacteria in basal compartment : (A) Salmonella; (B) B. fragilis; (C) impact of Salmonella and B. fragilis on TEER and (D) LY permeability after 21 days of culture. *p<0.05.

FIG. 3: Impact of B. fragilis or its cell free supernatant on Salmonella host cells interaction: (A) translocation of Salmonella; (B) evaluation of Salmonella growth (cfu/ml) during 3 h and 24 h of co-culture; (C) TEER evaluations on triple co-culture model; (D) mRNA expression changes in tight junction protein genes (occludin and ZO-1) in the triple coculture model infected by Salmonella Heidelberg, Bacteroides fragilis or both. *p<0.05.

FIG. 4: Impact of B. fragilis or its cell free supernatant on E. coli translocation after 3 h of incubation.

### EXAMPLE

### Material & Methods

### Cell Lines and Growth Culture

Caco-2 cells, obtained from American Type Culture Collection (ATCC), were cultivated in complete medium consisting of Dulbecco's modified Eagle medium (DMEM) supplemented with 20% fetal bovine serum, 1% L-glutamine and 1% penicillin and streptomycin. HT29-MTX cells were kindly provided by CRB CelluloNet (SFR Biosciences, CNRS UMS 3444, Inserm US 8, Université Claude-Bernard, Lyon, France) and were grown in the same medium as Caco-2 with only 10% of fetal bovine serum under a 5% CO2 water saturated atmosphere [17]. The Raji B (ECACC 85011429), issued from human Burkitt's lymphoma cell-line, were grown in RPMI 1640 medium supplemented with 10% fetal bovine serum, 1% non-essential amino acids, 1% L-glutamin and 1% penicillin and streptomycin, at 37° C. in a 5% CO2 water saturated atmosphere.

Upon confluence, cells were harvested with trypsin-EDTA and a predetermined amount of cells of each type were mixed prior to seeding to yield cell ratio of 9:1 (Caco-2:HT29-MTX) on the apical chamber of polycarbonate Transwell® inserts and maintained as described by Schimpel et al., 2014 [16]. After 14 days of culture, Raji B cells are added to the basolateral chamber to induce the differentiation of Caco-2 cells into M cells [16]. Caco-2/HT29-MTX and RajiB co-culture were maintained for 7 days in DMEM.

### TEER Measurements and Paracellular Permeability Study

The integrity of the polarized epithelial co-culture (Caco-2:HT29-MTX: M cells) was evaluated by measuring the transepithelial electrical resistance (TEER) using an Ohm/voltmeter (EVOM2; World Precision Instruments). The resistance obtained from a cell free culture insert was subtracted from resistance measured across each well and resistance values were calculated in Ohms (Ω)·cm2 by multiplying the resistance values by the filter surface area.

The integrity of polarized cells was checked also by measuring the Lucifer yellow (LY) transport rate. Regarding the paracellular permeability study, LY solution at 10 μM was prepared in DMEM, then added to the apical side of the insert while only DMEM was added in the basolateral side. After incubation for the periods indicated for TEER study, the solution in the basal compartment was collected and the fluorescence intensity of LY was measured using POLARstar Omega Microplate Reader. Results were expressed in pmol/cm2/s in kinetic differentiation or as percentage of LY permeability inhibition compared to insert without cells.

### Transmission Electron Microscopy

Transmission electron microscopy (TEM) was performed on polarized cells after 21 days of growth on polycarbonate Transwell® cell culture inserts. After several washing with PBS, samples were fixed for 2 hours in room temperature in 2.5% glutaraldehyde dissolved in 0.1 M cacodylate, postfixed in 1% osmium tetroxide for 1 hour at room temperature, rinsed in cacodylate buffer, and dehydrated in an ascending series of ethanol. The polycarbonate membrane contained in the inserts and on which the cells grown was recovered, then cut into thin strips. Samples were then infiltrated with an ascending concentration of Epon resin in ethanol mixtures. Finally, they were placed in fresh Epon for several hours and then embedded in Epon for 48 hours at 60° C. Resins blocks were sectioned into 80 nm ultrathin sections using LEICA UC7 ultramicrotome (LEICA Systems, Vienna, Austria): cut sections were performed so that it allowed to visualize transversally Transwell® membrane with cells layer. These sections were mounted on copper grids and stained. Grids were observed using a TEM JEOL-JEM 1400 (JEOL Ltd, Tokyo, Japan) at an accelerating voltage of 120 kV and equipped with a Gatan Inc. Orius 1000 camera.

### Quantification of Selected Genes Expression Level by Quantitative Reverse Transcription PCR (RT-qPCR)

After 21 days of co-culture, RNA were extracted from the upper chamber using Total RNA and Protein isolation kit (Macherey-Nagel) according to the manufacturer's instructions. Afterwards, High-Capacity cDNA Reverse Transcription Kit (Applied biosystems) was used to reverse-transcribe the RNA into cDNA. Then, the selected genes specific for each cells were relatively quantified using StepOnePlus (Applied Biosystems) with the SYBR Green PCR Master Mix (Applied Biosystems) [18]. Genes playing essential roles for each cells were selected: sucrase isomaltase (SI) which is specific of Caco-2, mucin-2 (muc2) secreted by HT29-MTX, and glycoprotein 2 (GP2) associated to M cells. Primers used for these selected genes were described in table 1. Each gene was normalized to the TBP (TATA box binding protein) mRNA expression level before calculation of the fold-change values. Relative gene expression was calculated by the 2-ΔΔCT method [19].

### Bacteria and Growth Conditions

Strain of Salmonella Heidelberg B182 (S. Heidelberg), with a hypermutator phenotype (deletion of 12 bp in mutS) was grown overnight at 37° C. as we previously described [18]. The non-toxigenic Bacteroides fragilis strain (B. fragilis), ATCC 25285 [20], [21], was purchased from the American Type Culture Collection. To mimic the in vivo scenario of gut lumen, S. Heidelberg and B. fragilis were applied simultaneously to the apical side of the Transwell® system at a ratio of 1:99 respectively. They were cultured in DMEM medium containing 20% SVF, 1% L-glutamin and 1% L-cystein. To separate supernatant and bacterial pellets, the media were centrifuged at 3000×g for 5 min. Supernatant samples were sterilized in 0, 22 μm filter. B. fragilis supernatant was used to evaluate its impact on S. Heidelberg growth and translocation.

### Evaluation of Bacterial Translocation

After 21 days of cellular culture, in order to study the cells/bacteria interaction, S. Heidelberg and B. fragilis after overnight culture were recovered and added on washed intestinal epithelial cell layer at an MOI of 10 and incubated at 37° C. for 3 h. Following incubation, basal and apical medium were separately collected and CFU enumeration was performed. The number of translocated bacteria recovered in the lower chambers was expressed as a ratio between this number and number of bacteria counted in the upper chamber.

### Statistical Analysis

Experiments were performed at least in triplicates and data were analyzed using Student's t-test. Data presented as mean±SD and p-value less than 0.05 was considered as significant.

### Results

### Characterization of Caco-2/HT29-MTX/M Cells Co-Culture

In this study, we developed an original in vitro triple co-culture model composed of enterocytes (Caco-2), goblets cells (HT29-MTX) secreting mucus and M cells. M cells were obtained by inducing the differentiation of enterocytes of the Caco-2 cell line cultured in close contact with B lymphocytes of the Raji B cell line already reported by Schimpel et al., 2014 [16].

We investigated the cell morphologies by transmission electron microscopy (TEM) in a triple co-culture of 21 days in a Transwell® membrane. TEM allowed to recognize M cells through the particular shape of their apical membrane exhibiting short and irregular microvilli, whereas Caco-2 cells showed a typical brush border (data not shown). Goblet cells, randomly distributed, were visualized by their typical mucus containing vesicles (data not shown). To further characterize the triple co-culture model, we checked the level of expression of genes that are molecular markers for each cell type. At the differentiation state (21 days), in the double (Caco-2/HT29-MTX) and triple (Caco-2/HT29-MTX/M) co-culture models, muc2 expression was significantly upregulated: 4, 36±1, 01 and 3, 9±0, 37 fold higher than Caco-2 alone respectively (data not shown). The gene expression of the M-like cells markers GP2 increased significantly when the co-cultures were grown with Raji-B cells in the basolateral chamber compared to Caco-2 alone (9.6±085 fold higher than Caco-2). Sucrase isomaltase (SI) mRNA relative expression was not significantly different between all conditions.

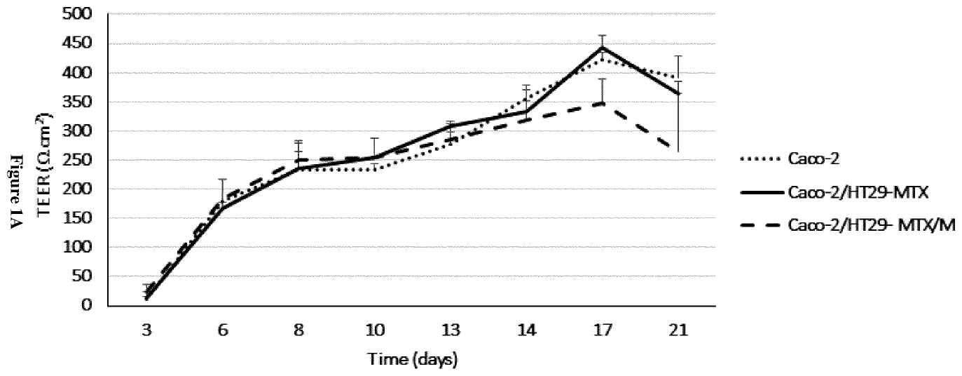

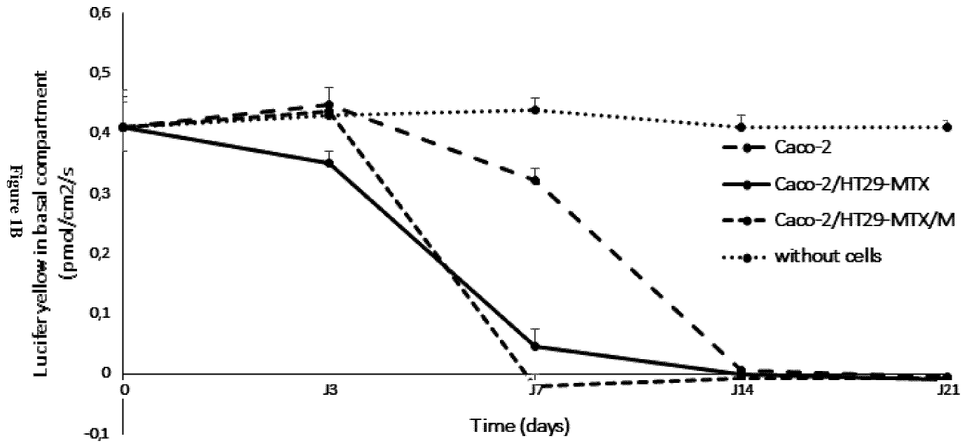

To investigate the permeability of this in vitro triple co-culture model, we evaluated the epithelial barrier integrity by measuring TEER. The TEER values increased with time for all cells (FIG. 1A). At the end of the 21 days of differentiation process, Caco-2 monoculture presented the highest value (390±37 Ω·cm2) followed by double co-culture (Caco-2/HT29-MTX) (364±20 Ω·cm2) while triple co-culture (Caco-2/HT29-MTX/RajiB) showed a value of 264±99 Ω·cm2. Those values were not significantly different from Caco-2 alone (p>0.05).

The cell permeability was investigated by measuring the paracellular efflux of a fluorescent tracer, Lucifer yellow (LY), across our models. After 7 days of culture, LY permeability decreased for all conditions. These results matched with TEER values as at 7 days.

After 21 days of differentiation, there was no detectable amount of LY in the basal chamber whereas in insert without cells, LY could be found (FIG. 1B).

### B. fragilis and S. Heidelberg Translocation Across Triple Co-Culture Model

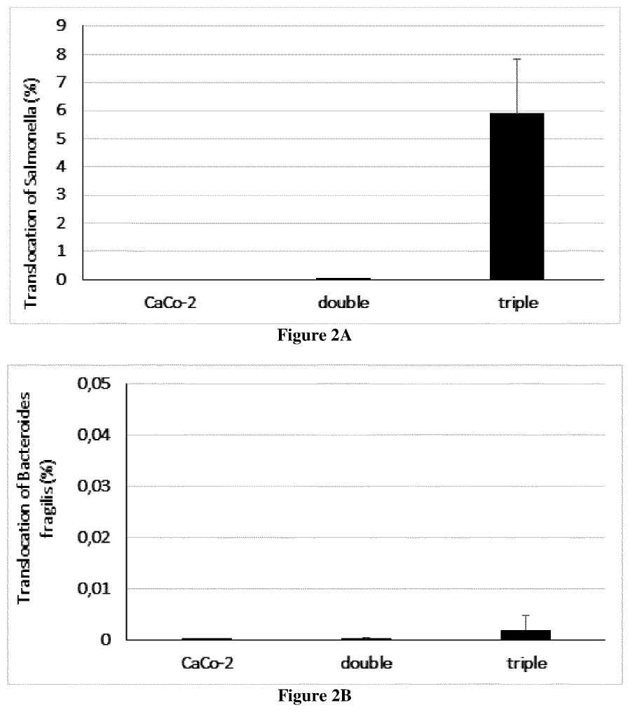

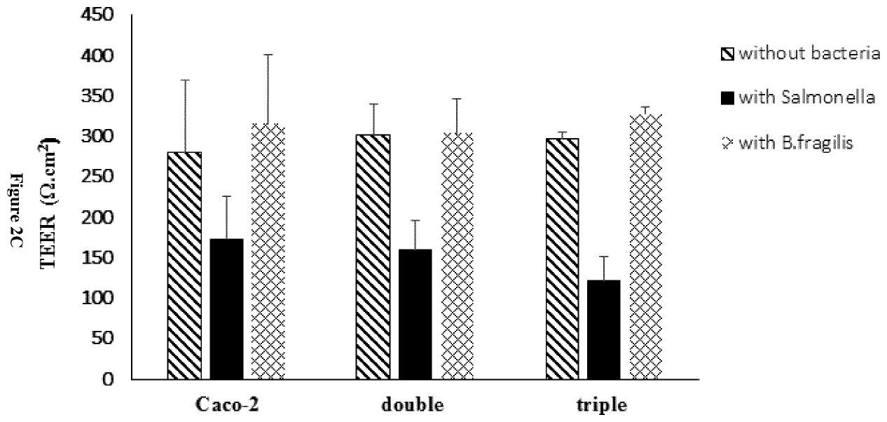

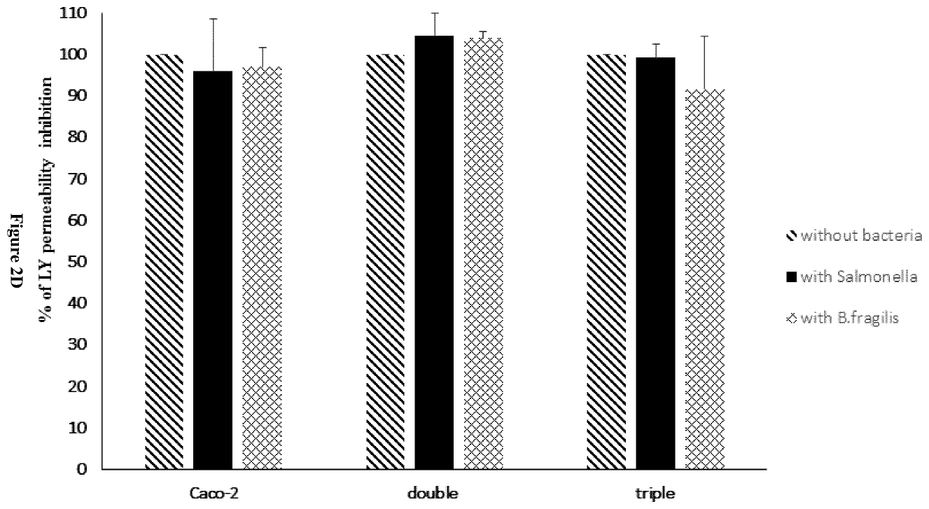

To evaluate the impact of M cells on bacteria translocation in the triple co-culture model, we evaluated the translocation rate of two different bacteria strains across this model. Those were a commensal, B. fragilis a non-toxigenic strain, and a pathogen, S. Heildeberg [9]. We compared data with measurement within the Caco-2 model and the double cell type model (Caco-2 and HT29-MTX without M Cells). For this purpose, we enumerated each strain of bacteria in basal compartment. S. Heidelberg translocated with the highest efficiency across triple co-culture model (5.9%±1.9) after 3 h of incubation whereas the translocation rate was 0.0003% ±0.00006 in Caco-2 alone and 0.002%±0.001 in double co-culture model (FIG. 2A). Concerning B. fragilis, the translocation was very weak through all cell models even in triple co-culture model (0.005%±0.006) (FIG. 2B).

The impact of bacteria exposure on the integrity of the different models was evaluated by measuring TEER (FIG. 2C). When double and triple co-culture models were exposed to B. fragilis, no significant modification of the TEER was shown (double and triple co-culture with a TEER of 303±42 Ω·cm2 and 327±9 Ω·cm2 respectively). In presence of S. Heidelberg, TEER decreased significantly (p<0.05) in double (160±36 Ω·cm2) and triple co-culture (121±30 Ω·cm2) compared to model without bacteria (302±36 Ω·cm2 and 296±9 Ω·cm2 respectively). In double co-culture model, TEER is of 160±36 Ω·cm2 but only 0.002%±0.001 of S. Heidelberg translocated in basal compartment whereas in triple co-culture model where TEER is 121±30 Ω·cm2, the translocation was of 5.9%±1.9.

Measuring the paracellular transport of LY across the different models infected by S. Heidelberg or B. fragilis, no significant increase of LY permeability was observed, compared to untreated cells (FIG. 2D). Thus, it appears that infection with S. Heidelberg significantly decreased the TEER of the triple model, but not enough to compromise the barrier integrity.

Enteric pathogens are known to perturb the intestinal epithelial barrier by modifying tight junctional proteins: zonula occludens (ZO) and occludin. Occludin and ZO-1 mRNA analysis showed that only occludin gene was significantly increased compared to uninfected cells in presence of S. Heidelberg. However, in presence of B. fragilis, the genes expression was the same as in the case of uninfected cells.

S. Heidelberg Translocation is Inhibited in Triple Co-Culture Model in Presence of B. fragilis

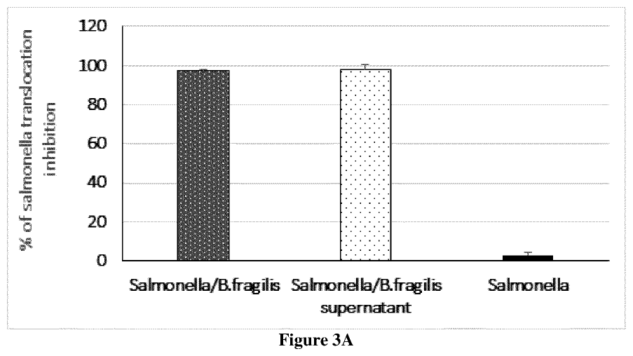

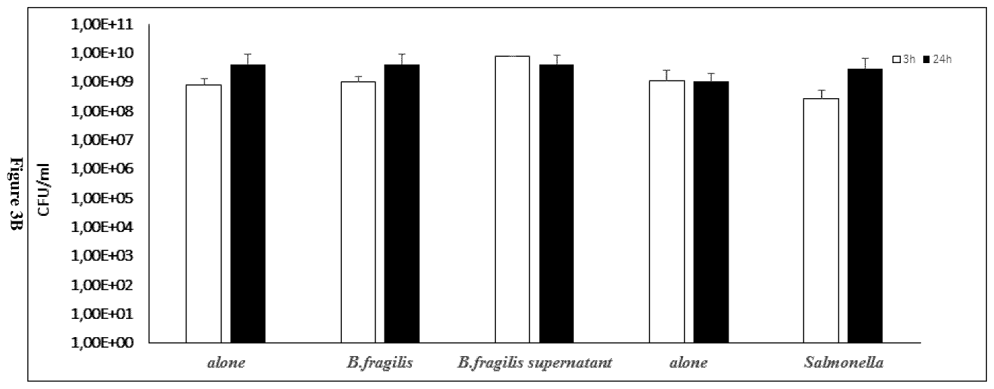

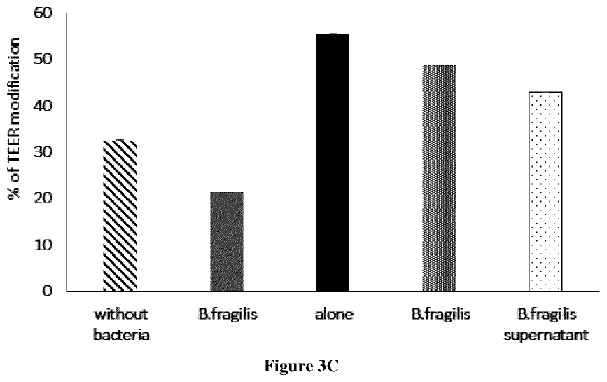

To evaluate whether B. fragilis can modify S. Heidelberg translocation rate, we mixed the two bacteria (S. Heidelberg/B. fragilis) in the upper chamber of the triple co-culture model. After a 3 h incubation, we quantified the presence of S. Heidelberg and B. fragilis in the basal compartment. We found that 89% of S. Heidelberg translocation was significantly (p<0.05) inhibited in presence of B. fragilis (FIG. 3A). Enumeration of each bacteria in the upper compartment at 3 h and 24 h (FIG. 3B) showed that there was no significant impact of B. fragilis on S. Heidelberg growth in the upper chamber. B. fragilis growth was also not impacted by the presence of S. Heidelberg (FIG. 3B).

Then, we analyzed the impact of a bacterial S. Heidelberg and B. fragilis co-culture on TEER and compared it to the model infected with only one of the bacteria (FIG. 3C). TEER in triple co-culture model infected by both bacteria was the same as when the co-cultures were exposed to S. Heidelberg alone and was lower than in cells infected by B. fragilis alone. The LY transfer rate was not modified in this condition (data not shown). Occludin and ZO-1 expression were the same as when this model was infected with S. Heidelberg alone (FIG. 3D).

### B. fragilis Supernatant Inhibited S. Heidelberg Translocation

In order to study the role of bacterial secreted substances on the inhibition of S. Heidelberg translocation, we examined whether B. fragilis supernatant could abrogate the effects of S. Heidelberg on intestinal cells. Firstly, we explored whether treatment with B. fragilis supernatant could reduce S. Heidelberg growth. The results presented in FIG. 3B showed that B. fragilis supernatant did not affect the growth of S. Heidelberg. When triple co-culture model was exposed to S. Heidelberg mixed to B. fragilis supernatant, a significant inhibition of Salmonella translocation was shown (FIG. 3A) without significantly affecting TEER or occludin gene expression (FIG. 3C). The level of S. Heidelberg translocation inhibition by B. fragilis supernatant was in the same range that the inhibition observed when S. Heidelberg was mixed with B. fragilis (86% and 89% respectively).

### B. fragilis and Its Cell Free Supernatant Inhibited Other Enterobacteriae Translocation Such as E. coli

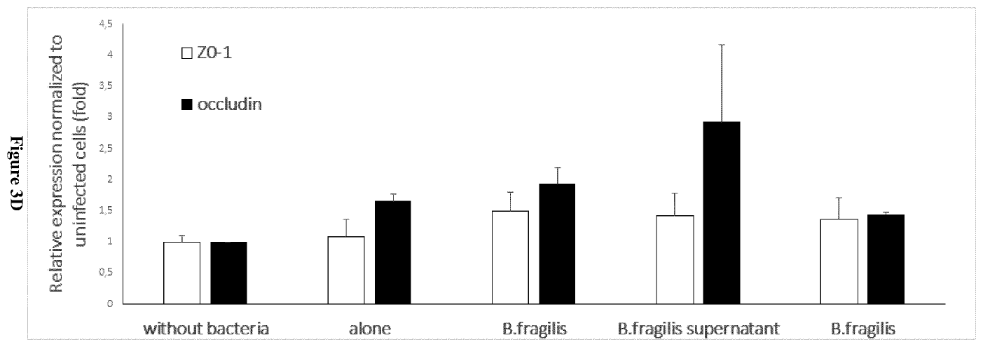

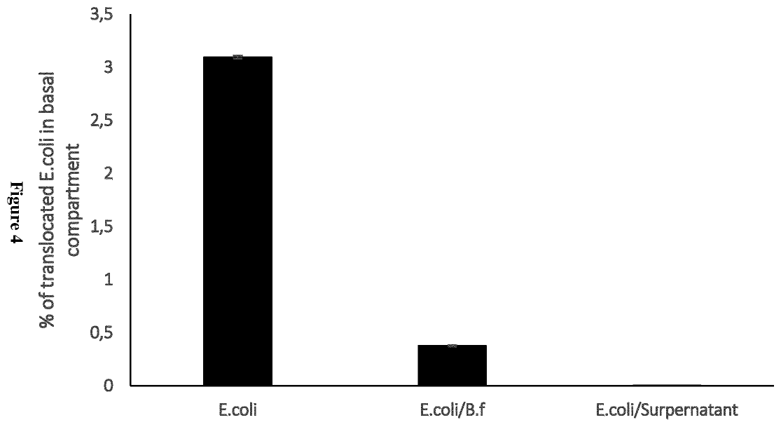

In order to investigate if B. fragilis or its cell free supernatant impact translocation of other enterobacteria, we have used Escherichia coli (E. coli ATCC11775). When triple co-culture model was exposed to E. coli mixed to B. fragilis or its free supernatant, an inhibition of E. coli translocation was shown (FIG. 4).

### Conclusion

By using an original triple co-culture model including Caco-2 cells (representing human enterocytes), HT29-MTX (representing mucus-secreting goblet cells), and M cells differentiated from Caco-2 by addition of Raji B lymphocytes, bacterial translocation was evaluated. The data showed that S. Heidelberg could translocate in the triple co-culture model with high efficiency, whereas for B. fragilis a weak translocation was obtained. When cells were exposed to both bacteria, S. Heidelberg translocation was inhibited. The cell-free supernatant of B. fragilis also inhibited S. Heidelberg translocation without impacting epithelial barrier integrity. This supernatant did not affect the growth of S. Heidelberg, demonstrating that the effects of growth (i.e. increasing number of bacteria over the time) and the effects of translocation (i.e. passage of bacteria across the intestinal epithelium) have to be differentiated in this study. The non-toxigenic B. fragilis confers health benefits to the host by reducting bacterial translocation.

### REFERENCES

Throughout this application, various references describe the state of the art to which this invention pertains. The disclosures of these references are hereby incorporated by reference into the present disclosure.

1. Nazli A, Wang A, Steen O, et al. Enterocyte cytoskeleton changes are crucial for enhanced translocation of nonpathogenic Escherichia coli across metabolically stressed gut epithelia. Infect Immun. 2006; 74(1):192-201.

2. Nazli A, Yang P-C, Jury J, et al. Epithelia under metabolic stress perceive commensal bacteria as a threat. Am J Pathol. 2004; 164(3):947-957.

3. Rios D, Wood M B, Li J, Chassaing B, Gewirtz A T, Williams I R. Antigen sampling by intestinal M cells is the principal pathway initiating mucosal IgA production to commensal enteric bacteria. Mucosal Immunol. 2016; 9(4):907-916.

4. Sansonetti P J, Arondel J, Cantey J R, Prévost M C, Huerre M. Infection of rabbit Peyer's patches by Shigella flexneri: effect of adhesive or invasive bacterial phenotypes on follicle-associated epithelium. Infect Immun. 1996; 64(7):2752-2764.

5. Clark M A, Hirst B H, Jepson M A. M-cell surface betal integrin expression and invasin-mediated targeting of Yersinia pseudotuberculosis to mouse Peyer's patch M cells. Infect Immun. 1998; 66(3):1237-1243.

6. Westphal S, Lügering A, Wedel J von, et al. Resistance of chemokine receptor 6-deficient mice to Yersinia enterocolitica infection: evidence of defective M-cell formation in vivo. Am J Pathol. 2008; 172(3):671-680.

7. Wilmshurst P, Sutcliffe H. Splenic abscess due to Salmonella Heidelberg. Clin Infect Dis Off Publ Infect Dis Soc Am. 1995; 21(4):1065.

8. Le Gall S, Desbordes L, Gracieux P, et al. Distribution of mutation frequencies among Salmonella enterica isolates from animal and human sources and genetic characterization of a Salmonella Heidelberg hypermutator. Vet Microbiol. 2009; 137(3-4):306-312.

9. Le Gall-David S, Zenbaa N, Bouchard D, et al. Hypermutator Salmonella Heidelberg induces an early cell death in epithelial cells. Vet Microbiol. 2015; 180(1-2):65-74.

10. Blázquez J. Hypermutation as a factor contributing to the acquisition of antimicrobial resistance. Clin Infect Dis Off Publ Infect Dis Soc Am. 2003; 37(9):1201-1209.

11. Sekirov I, Russell S L, Antunes L C M, Finlay B B. Gut microbiota in health and disease. Physiol Rev. 2010; 90(3):859-904.

12. Fijan S. Microorganisms with claimed probiotic properties: an overview of recent literature. Int J Environ Res Public Health. 2014; 11(5):4745-4767.

13. FolignéB, Daniel C, Pot B. Probiotics from research to market: the possibilities, risks and challenges. Curr Opin Microbiol. 2013; 16(3):284-292.

14. Wexler H M. Bacteroides: the good, the bad, and the nitty-gritty. Clin Microbiol Rev. 2007; 20(4):593-621.

15. Li Z, Deng H, Zhou Y, et al. Bioluminescence Imaging to Track Bacteroides fragilis Inhibition of Vibrio parahaemolyticus Infection in Mice. Front Cell Infect Microbiol. 2017; 7:170.

16. Schimpel C, Teubl B, Absenger M, et al. Development of an advanced intestinal in vitro triple culture permeability model to study transport of nanoparticles. Mol Pharm. 2014; 11(3): 808-818.

17. Lesuffleur T, Kornowski A, Augeron C, et al. Increased growth adaptability to 5-fluorouracil and methotrexate of HT-29 sub-populations selected for their commitment to differentiation. Int J Cancer. 1991; 49(5):731-737.

18. Le Bars H, Le Gall-David S, Renoux V M, Bonnaure-Mallet M, Jolivet-Gougeon A, Bousarghin L. Impact of a mutator phenotype on motility and cell adherence in Salmonella Heidelberg. Vet Microbiol. 2012; 159(1-2):99-106.

19. Livak K J, Schmittgen T D. Analysis of relative gene expression data using real-time quantitative PCR and the 2(-Delta Delta C(T)) Method. Methods San Diego Calif. 2001; 25(4):402-408.

20. Choi V M, Herrou J, Hecht A L, et al. Activation of Bacteroides fragilis toxin by a novel bacterial protease contributes to anaerobic sepsis in mice. Nat Med. 2016; 22(5):563-567.

21. Sears C L, Geis A L, Housseau F. Bacteroides fragilis subverts mucosal biology: from symbiont to colon carcinogenesis. J Clin Invest. 2014; 124(10):4166-4172.

22. Albac S, Schmitz A, Lopez-Alayon C, et al. Candida albicans is able to use M cells as a portal of entry across the intestinal barrier in vitro: C. albicans interactions with M cells. Cell Microbiol. 2016; 18(2):195-210.

23. Garcia-Rodriguez A, Vila L, Cortés C, Hernández A, Marcos R. Exploring the usefulness of the complex in vitro intestinal epithelial model Caco-2/HT29/Raji-B in nanotoxicology. Food Chem Toxicol. 2018; 113:162-170.

24. Roberts C L, Keita A V, Duncan S H, et al. Translocation of Crohn's disease Escherichia coli across M-cells: contrasting effects of soluble plant fibres and emulsifiers. Gut. 2010; 59(10):1331-1339.

25. Steffen E K, Berg R D, Deitch E A. Comparison of translocation rates of various indigenous bacteria from the gastrointestinal tract to the mesenteric lymph node. J Infect Dis. 1988; 157(5):1032-1038.

26. Ohno H. Intestinal M cells. J Biochem (Tokyo). 2016; 159(2):151-160.

27. Lapthorne S, Macsharry J, Scully P, Nally K, Shanahan F. Differential intestinal M-cell gene expression response to gut commensals. Immunology. 2012; 136(3):312-324.

28. Köhler H, Sakaguchi T, Hurley B P, Kase B J, Reinecker H-C, McCormick B A. Salmonella enterica serovar Typhimurium regulates intercellular junction proteins and facilitates transepithelial neutrophil and bacterial passage. Am J Physiol-Gastrointest Liver Physiol. 2007; 293(1):G178—G187.

29. Sears C L. Molecular Physiology and Pathophysiology of Tight Junctions V. Assault of the tight junction by enteric pathogens. Am J Physiol-Gastrointest Liver Physiol. 2000; 279(6):G1129—G1134.

30. Yu Q, Wang Z, Yang Q. Lactobacillus amylophilus D14 protects tight junction from enteropathogenic bacteria damage in Caco-2 cells. J Dairy Sci. 2012; 95(10):5580-5587.

31. Shao Y-X, Lei Z, Wolf PG, Gao Y, Guo Y-M, Zhang B-K. Zinc Supplementation, via GPR39, Upregulates PKCζ to Protect Intestinal Barrier Integrity in Caco-2 Cells Challenged by Salmonella enterica Serovar Typhimurium. J Nutr. 2017; 147(7):1282-1289.

32. Le Bars H, Bousarghin L, Bonnaure-Mallet M, Jolivet-Gougeon A, Barloy-Hubler F. Complete genome sequence of the strong mutator Salmonella enterica subsp. enterica serotype Heidelberg strain B182. J Bacteriol. 2012; 194(13):3537-3538.

33. Zhang W, Zhu B, Xu J, et al. Bacteroides fragilis Protects Against Antibiotic-Associated Diarrhea in Rats by Modulating Intestinal Defenses. Front Immunol [Internet]. 2018 [cited 2019 Feb. 20]; 9. Available from: http://journal.frontiersin.org/article/10.3389/fimmu.2018.01040/full

34. Chatzidaki-Livanis M, Coyne M J, Comstock L E. An antimicrobial protein of the gut symbiont Bacteroides fragilis with a MACPF domain of host immune proteins: Secreted antimicrobial protein of B. fragilis. Mol Microbiol. 2014; 94(6):1361-1374.

35. Koropatkin N M, Cameron E A, Martens E C. How glycan metabolism shapes the human gut microbiota. Nat Rev Microbiol. 2012; 10(5):323-335.

36. Hentges D J, Maier B R. Inhibition of Shigella flexneri by the Normal Intestinal Flora III. Interactions with Bacteroides fragilis Strains in Vitro. Infect Immun. 1970; 2(4):364-370.

37. Fong F L Y, Kirjavainen P V, El-Nezami H. Immunomodulation of Lactobacillus rhamnosus GG (LGG)-derived soluble factors on antigen-presenting cells of healthy blood donors. Sci Rep [Internet]. 2016 [cited 2019 Feb. 25]; 6(1). Available from: http://www.nature.com/articles/srep22845

38. Delday, M., Mulder, I., Logan, E. T., Grant, G., 2019. Bacteroides thetaiotaomicron Ameliorates Colon Inflammation in Preclinical Models of Crohn's Disease. Inflammatory Bowel Diseases 25, 85-96. https://doi.org/10.1093/ibd/izy281

39. Hosseini, M., Kolangi Miandare, H., Hoseinifar, S. H., Yarahmadi, P., 2016. Dietary Lactobacillus acidophilus modulated skin mucus protein profile, immune and appetite genes expression in gold fish (Carassius auratus gibelio). Fish & Shellfish Immunology 59, 149-154. https://doi.org/10.1016/j.fsi.2016.10.026

40. Jiang, Y., Zhou, S., Chu, W., 2019. The effects of dietary Bacillus cereus QSI-1 on skin mucus proteins profile and immune response in Crucian Carp (Carassius auratus gibelio). Fish & Shellfish Immunology 89, 319-325. https://doi.org/10.1016/ifsi.2019.04.014

41. Li, K., Petersen, G., Barco, L., Hvidtfeldt, K., Liu, L., Dalsgaard, A., 2017. Salmonella Weltevreden in integrated and non-integrated tilapia aquaculture systems in Guangdong, China. Food Microbiology 65, 19-24. https://doi.org/10.1016/j.fm.2017.01.014

42. Roeselers, G., Mittge, E. K., Stephens, W. Z., Parichy, D. M., Cavanaugh, C. M., Guillemin, K., Rawls, J. F., 2011. Evidence for a core gut microbiota in the zebrafish. ISME J 5, 1595-1608. https://doi.org/10.1038/ismej.2011.38

43. Su, X., Yin, X., Liu, Y., Yan, X., Zhang, S., Wang, X., Lin, Z., Zhou, X., Gao, J., Wang, Z., Zhang, Q., 2020. Gut Dysbiosis Contributes to the Imbalance of Treg and Th17 Cells in Graves' Disease Patients by Propionic Acid. The Journal of Clinical Endocrinology & Metabolism 105, dgaa511.https://doi.org/10.1210/clinem/dgaa511

44. Yuan, S., Shen, J., 2021. Bacteroides vulgatus diminishes colonic microbiota dysbiosis ameliorating lumbar bone loss in ovariectomized mice. Bone 142, 115710. https://doi.org/10.1016/j.bone.2020.115710

1. A method of treating a diseases caused by Enterobacteriae in a subject in need thereof comprising,
administering to the subject a therapeutically effective amount of a Bacteroides fragilis strain.

2. The method according to claim 1 wherein the Enterobacteriae is a Salmonella selected from the group consisting of Salmonella Heidelberg, Escherichia coli, Yersinia pestis, Klebsiella and Shigella.

3. The method according to claim 2 wherein the Enterobacteriae is a Salmonella or Escherichia coli.

4. The method according to claim 1, wherein the Bacteroides fragilis strain is administered as a probiotic.

5. The method according to claim 1, wherein the disease caused by Enterobacteriae is Salmonellosis, typhoid fever, diarrhea Crohn's disease, travelers' diarrhea or ulcerative colitis.

6. The method according claim 1, wherein the subject is an animal.

7. The method according to claim 6 wherein the animal is a fish.

8. A method of treating an Inflammatory Bowel Disease and/or an Irritable Bowel Syndrome in a subject in need thereof comprising,
administering to the subject a therapeutically effective amount of a Bacteroides fragilis strain.

9. The method according to claim 8, wherein the Inflammatory Bowel Disease is Crohn's disease or ulcerative colitis.

10. The method according to claim 1, wherein the Bacteroides fragilis strain is a non-toxigenic strain.

11. A therapeutic composition comprising a Bacteroides fragilis strain.

12-13. (canceled)
